(19) United States
(12) Patent Application Publication
(10) Pub. No.: US 2015/0031984 A1
(43) Pub. Date: Jan. 29, 2015

(54) Magnetic Resonance Device having a Projection Unit

(71) Applicant: Daniel Driemel, Oederan (DE)

(72) Inventor: Daniel Driemel, Oederan (DE)

(21) Appl. No.: 14/327,684

(22) Filed: Jul. 10, 2014

(30) Foreign Application Priority Data
Jul. 23, 2013 (DE) 102013214390.7

(51) Int. Cl. A61B 5/00; G01R 33/48; A61B 5/055

(52) U.S. Cl. CPC A61B 5/742; A61B 5/0555; A61B 5/4064; G01R 33/4806
USPC 600/415; 600/410

(57) ABSTRACT

The embodiments include a magnetic resonance device having a head antenna unit configured for acquiring magnetic resonance signals, and a projection unit that has a presentation surface unit for presenting picture elements and a first projection surface unit for presenting a projection of the picture elements. The first projection surface unit has a center and the presentation surface unit has a center, where the center of the first projection surface unit defines an axis together with the center of the presentation surface unit. The projection unit has a second projection surface unit, a first mirror plane for projecting a picture element onto the second projection surface unit and a second mirror plane for projecting a picture element onto the first projection surface unit, the axis intersecting a normal vector of the first mirror plane and/or a normal vector of the second mirror plane.

### CROSS-REFERENCE TO RELATED APPLICATIONS

This application claims the benefit of DE 10 2013 214390.7, filed on Jul. 23, 2013, which is hereby incorporated by reference in its entirety.

### BACKGROUND

Neurological examinations are one of the main applications of magnetic resonance tomography, where such examinations may be carried out by functional magnetic resonance imaging. This entails stimulating areas of the patient's brain by visual or acoustic stimulation, and visualizing and identifying the respective active areas in the brain by functional magnetic resonance imaging.

Among the embodiments used for visual stimulation of the patient is a mirror that enables the patient to view a monitor, the monitor being arranged at a head-end region of a patient receiving zone of a magnetic resonance device. In this case, images or texts are presented for stimulation purposes. However, projecting images and/or texts from the monitor onto the mirror that is arranged above the patient's head leads to laterally reversed images and texts being represented on the mirror, which is problematic in particular for the presentation of texts.

### SUMMARY AND DESCRIPTION

The object underlying the present embodiments is, in particular, to realize a true, non-inverted presentation of images and/or texts for stimulating a patient during a magnetic resonance imaging session.

The embodiments are based on a magnetic resonance device having a head antenna unit that is configured for acquiring magnetic resonance signals during a magnetic resonance measurement, and having a projection unit that has a presentation surface unit for presenting picture elements and a first projection surface unit for presenting a projection of the picture elements presented on the presentation surface unit. The first projection surface unit has a center and the presentation surface unit has a center, the center of the first projection surface unit defining an axis with the center of the presentation surface unit.

It is proposed that the projection unit have a second projection surface unit, a first mirror plane for projecting a picture element from the presentation surface unit onto the second projection surface unit, and a second mirror plane for projecting a picture element from the second projection surface unit onto the first projection surface unit, where the axis intersects a normal vector of the first mirror plane and/or a normal vector of the second mirror plane. In this way, it is advantageously possible to generate a realistic and/or true, non-inverted image on the first projection surface unit. The image may be arranged in a field of view and/or visual range of the patient for, in particular, a visual stimulation of the patient during functional magnetic resonance imaging. Texts may be presented so as to be readable for the patient by the true and/or realistic, non-inverted image, with the result that in addition a relaxed and stress-free viewing of the projected picture elements may be made possible for the patient.

The presentation surface unit may include a monitor that is arranged at an end region, in particular, a head-end region, of a patient receiving zone of the magnetic resonance device. By the projection unit picture elements that are presented on the monitor are projected onto the first projection surface unit in order to present the picture elements for the patient in an examination position during the magnetic resonance imaging examination. Each unit of the individual projection surface units advantageously includes a projection surface, which is embodied at least in part as a reflecting surface, for projecting and/or mirroring the picture elements that are to be presented. In this arrangement, the reflecting surfaces of the individual projection surface units may be oriented facing toward a facial region and/or facial opening of the head coil unit so that the view of a patient arranged inside the head coil unit is directed directly onto at least one of the projection surface units during the magnetic resonance imaging examination. The center of the presentation surface unit may be defined by a center of a presentation surface of the presentation surface unit. The center of the first projection surface unit may be defined by a center of a projection surface, in particular, of a reflecting surface of the first projection surface unit. The first mirror plane and/or the second mirror plane may be formed by a center plane and/or a mirror plane of symmetry, where the presentation surface of the display unit is arranged with respect to the first mirror plane symmetrically to the second projection surface of the second projection surface unit, and the second projection surface of the second projection surface unit is arranged with respect to the second mirror plane symmetrically to the first projection surface of the first projection surface unit.

It is furthermore proposed that the first projection surface unit have a first projection surface and the first projection surface have an angle of inclination of 45° with respect to a reclining surface of the head antenna unit and/or a reclining surface of a patient positioning device. This embodiment of the first projection surface unit enables a projected image on the first projection surface that stands upright from a viewing direction of the patient and in this way an easily viewable image may be generated for the patient positioned inside the head antenna unit. Furthermore, a relaxed and stress-free viewing of the projected picture elements may be made possible for the patient owing to the upright and true, non-inverted projection of the picture elements. The first projection surface may have an angle of inclination of 45° with respect to the presentation surface in a projection onto a sagittal plane of the head antenna unit.

Alternatively, or in addition, the first projection surface of the first projection surface unit may also have an angle of inclination of 45° with respect to a presentation surface of the display unit, thereby likewise generating a projected image on the first projection surface. The projected image stands upright from a viewing direction of the patient, and in this way, an easily viewable image may be generated for the patient positioned inside the head antenna unit. The presentation surface of the presentation surface unit may be arranged substantially perpendicularly to a reclining surface of the patient positioning device.

It is furthermore proposed that the magnetic resonance device have a patient positioning device having a reclining surface, where the axis together with a directional vector that is aligned perpendicularly to a reclining surface of the head antenna unit and/or perpendicularly to the reclining surface of the patient positioning device spans a plane and the second projection surface unit is arranged to the side of the plane. In this way, it is possible to achieve an advantageous rotation of a laterally reversed projection on a second projection surface of the second projection surface unit to a true, non-inverted projection onto the first projection surface of the first projection surface unit. In this case a good visibility of the first projection surface of the first projection surface unit may furthermore be made possible for the patient, since the second projection surface unit may be arranged outside a field of view of the patient. What is to be understood by an arrangement to the side in this context is in particular an arrangement of the second projection surface unit that may be arranged completely alongside the plane.

In an advantageous development, it is proposed that the first projection surface unit be embodied separately from the second projection surface unit. This enables a projection of a projected picture element to be presented uniquely for the patient. The first projection surface unit of the projection unit may be arranged in a field of view of the patient positioned inside the head antenna unit.

In a further embodiment, it is proposed that the first projection surface unit have a first projection surface and the second projection surface unit have a second projection surface, the first projection surface including an angle of inclination of 67.5° with respect to the second projection surface. In certain embodiments, it is advantageously possible to generate a realistic and/or true, non-inverted image on the first projection surface of the first projection surface unit for in particular a visual stimulation of the patient during functional magnetic resonance imaging so that texts may be presented in readable form for the patient. Furthermore, a relaxed and stress-free viewing of the projected picture elements may be made possible for the patient in this way. In certain embodiments, an exact setting of the angle of inclination of precisely 67.5° between the first projection surface and the second projection surface is required for the true, non-inverted imaging of picture elements.

It is furthermore proposed that the head antenna unit have a facial opening and a median plane, the center of the first projection surface unit being arranged spaced at a distance apart from the facial opening of the head antenna unit in the median plane. As a result hereof the first projection surface unit, in particular, the first projection surface, is advantageously arranged centrally in a field of view of a patient positioned inside the head antenna unit, such that a good visibility of the first projection surface of the first projection surface unit is given for the patient for a visual stimulation for the purpose of a functional magnetic resonance imaging examination. Furthermore, it is advantageously possible in this way to prevent undesirable movements of the patient's head for viewing the first projection surface of the first projection surface unit during the functional magnetic resonance imaging examination. What is to be understood in this context by the median plane of the head antenna unit is, in particular, a plane that divides the head antenna unit into precisely a right half and a left half, the median plane in this case being aligned perpendicularly to the facial opening of the head antenna unit. In particular, the median plane of the head antenna unit is formed by a mirror plane of symmetry of the head antenna unit that divides the head antenna unit into two mutually symmetrical halves or subsections. Furthermore, the median plane of the head antenna unit may be substantially identical to a sagittal plane of the patient positioned inside the head antenna unit.

In an advantageous development, it is proposed that the head antenna unit have a facial opening and a sagittal plane and that the first projection surface unit and the second projection surface unit be arranged directly adjacent to each other, a boundary line between the first projection surface unit and the second projection surface unit being arranged spaced at a distance apart from the facial opening of the head antenna unit in the sagittal plane. This enables a compact and space-saving projection unit to be provided for a functional magnetic resonance imaging examination on a patient. By this embodiment of the projection unit, it is furthermore advantageously possible to reduce a risk of collision of the projection unit with a housing enclosing the patient receiving zone during an operation to introduce the patient positioning device together with the patient and the projection unit into the patient receiving zone.

Advantageously, the first projection surface unit has a first projection surface and the second projection surface unit has a second projection surface, the first projection surface including an angle of 90° with the second projection surface. This arrangement of the two projection surfaces relative to each other enables an upright and realistic and/or true, non-inverted presentation on the first projection surface unit, such that texts may be presented in readable form for the patient. It is furthermore possible in this way to make a relaxed and stress-free viewing of the projected picture elements possible for the patient. In certain embodiments, an exact setting of the angle of precisely 90° between the first projection surface and the second projection surface is necessary for the true, non-inverted imaging of picture elements.

A space-saving arrangement of the presentation surface unit inside the magnetic resonance device may advantageously be achieved if the magnetic resonance device has a patient positioning device and/or a patient receiving zone. The presentation surface unit is arranged at an end region of the patient positioning device and/or at an end region of the patient receiving zone. In this case, the presentation surface unit is advantageously arranged at a head-end region of the patient positioning device and/or of the patient receiving zone, such that an obstruction caused by the arrangement of the presentation surface unit during a positioning operation of the patient on the patient positioning device and/or inside the patient receiving zone may advantageously be prevented.

In an advantageous development, it is proposed that the projection unit have a retaining device for positioning the first projection surface unit and/or the second projection surface unit. The retaining device is arranged at the head antenna unit. It is possible to achieve a space-saving and compact arrangement of the individual projection surface units during the magnetic resonance imaging examination inside the patient receiving zone of the magnetic resonance device. Furthermore, an undesirable collision of the projection unit with the head antenna unit during an operation to introduce the patient together with the head antenna unit into the patient receiving zone of the magnetic resonance device may advantageously be prevented in the process. In such an arrangement, the retaining device may have one or more retaining elements, (e.g., retaining arms), for positioning the first projection surface unit and/or for positioning the second projection surface unit.

Alternatively, or in addition, the magnetic resonance device has a patient positioning device, the projection unit having a retaining device for positioning the first projection surface unit and/or the second projection surface unit and the retaining device being arranged on the patient positioning device. A compact arrangement of the positioning unit inside the patient receiving zone of the magnetic resonance device may similarly be achieved in this way. Furthermore, an undesirable collision of the projection unit with the patient positioning device and/or with the head antenna unit during an operation to introduce the patient together with the head antenna unit into the patient receiving zone of the magnetic resonance device may also advantageously be prevented in the process.

An advantageous adjustment and/or positioning of the projection unit, in particular, with regard to a patient's body size and/or anatomy, may advantageously be achieved if the retaining device is movably arranged on the head antenna unit and/or on the patient positioning device. This furthermore enables the patient to be positioned on the patient positioning device and/or a head region of the patient to be positioned inside the head antenna unit in a collision-free manner with the projection unit, thereby minimizing a risk of injury to the patient. A displacement may take place along a longitudinal extension of the patient positioning device, an alignment and/or orientation of the two projection surface units relative to each other being preserved owing to the displacement of the retaining device. Alternatively, or in addition, the retaining device may also be arranged on the patient positioning device and/or the head antenna unit so as to be rotatable, such that the projection surface units may be rotated out of a positioning range and/or reclining range of the patient positioning device and/or of the head antenna unit. A position of the projection surface units may be signaled to a member of the medical operating staff, for example, by an end position and/or a locking position of the retaining device on the patient positioning device and/or the head antenna unit, where, in the position, the projection surface units have an alignment and/or orientation relative to each other and/or with respect to the presentation surface unit that allows an upright and true, non-inverted imaging of picture elements on the first projection surface unit.

It is furthermore proposed that the retaining device have at least one adjusting element for adjusting a position of the first projection surface unit and/or of the second projection surface unit. In certain embodiments, a setting of a projection surface of the projection surface unit may be adjusted to an examination position of the patient and/or to a viewing direction of the patient. The adjusting element may be for example an articulated-joint unit; in particular, a ball-and-socket joint unit, and/or a flexible stand, an adjustment and/or fixing may be effected by way of a clamping action of the adjusting element such that a simple and quick adjustment and/or fixing may be realized.

The scope of the present invention is defined solely by the appended claims and is not affected to any degree by the statements within this summary. The present embodiments may obviate one or more of the drawbacks or limitations in the related art.

### BRIEF DESCRIPTION OF THE DRAWINGS

FIG. 1 depicts a schematic representation of an embodiment of a magnetic resonance device having a projection unit.

FIG. 2 depicts a schematic representation of an embodiment of a first arrangement of individual projection surface units of the projection unit relative to each other.

FIG. 3 depicts a schematic representation of a first embodiment of a retaining device for the first arrangement of the projection unit.

FIG. 4 depicts a schematic representation of a second embodiment of a retaining device for the first arrangement of the projection unit in a schematic representation.

FIG. 5 depicts a schematic representation of an embodiment of a second arrangement of individual projection surface units of the projection unit relative to each other.

FIG. 6 depicts a schematic representation of a first embodiment of a retaining device for the second arrangement of the projection unit.

FIG. 7 depicts a schematic representation of a second embodiment of a retaining device for the second arrangement of the projection unit.

### DETAILED DESCRIPTION

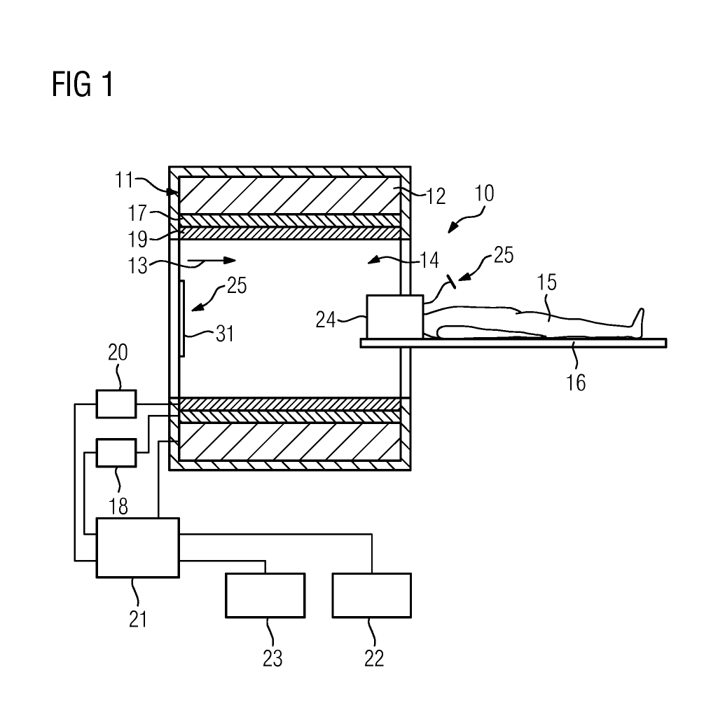

FIG. 1 depicts a magnetic resonance device 10 in a schematic representation. The magnetic resonance device 10 includes a magnet unit 11 having a main magnet 12 for generating a strong and in particular constant main magnetic field 13. The magnetic resonance device 10 additionally has a patient receiving zone 14 in the shape of a cylinder for receiving a patient 15, the patient receiving zone 14 being cylindrically surrounded by the magnet unit 11 in a circumferential direction. The patient 15 may be introduced into the patient receiving zone 14 by a patient positioning device 16 of the magnetic resonance device 10. The patient positioning device 16 is movably arranged inside the patient receiving zone 14 of the magnetic resonance device 10 for this purpose.

The magnet unit 11 additionally has a gradient coil unit 17 for generating magnetic field gradients that is used for spatial encoding during imaging. The gradient coil unit 17 is controlled by a gradient control unit 18 of the magnetic resonance device 10. The magnet unit 11 furthermore includes a radiofrequency antenna unit 19 and a radiofrequency antenna control unit 20 intended for exciting a polarization that becomes established in the main magnetic field 13 generated by the main magnet 12. The radiofrequency antenna unit 19 is controlled by the radiofrequency antenna control unit 20 and radiates radiofrequency magnetic resonance sequences into an examination space that is substantially formed by the patient receiving zone 14.

In order to control the main magnet 11 of the gradient coil unit 18, and in order to control the radiofrequency antenna control unit 20, the magnetic resonance device 10 has a control unit 21 formed by a computing unit. The control unit 21 centrally controls the magnetic resonance device 10, e.g., in order to execute a predetermined imaging gradient echo sequence. The control unit 21 furthermore includes an evaluation unit for evaluating image data. Control information, such as imaging parameters as well as reconstructed magnetic resonance images, may be displayed on a display unit 22, (e.g., on at least one monitor), of the magnetic resonance device 10 for an operator. The magnetic resonance device 10 additionally has an input unit 23 by which information and/or parameters may be input by an operator during a measurement procedure.

For functional magnetic resonance imaging purposes, in particular, for neurological examinations on the patient, the magnetic resonance device 10 also has a local head antenna unit 24 and a projection unit 25. For a head examination, the local head antenna unit 24 is arranged around the head of the patient 15 for the purpose of the pending magnetic resonance examination. The local head antenna unit 24 has a housing 26 having a helmet-like shape. The housing 26 includes a positioning surface and a reclining surface that are arranged on the same housing section of the head antenna unit 24. In this arrangement, the reclining surface includes a surface facing toward a receiving zone 29 of the head antenna unit 24, and the positioning surface includes a surface facing away from the receiving zone 29 of the head antenna unit 24. The reclining surface is provided as an aid to positioning the head of the patient 15 for the purpose of a magnetic resonance examination. The head antenna unit 24 is positioned on the patient positioning device 16 by the positioning surface. The local head antenna unit 24 additionally has a facial opening 30 from which the face of the patient 15 protrudes when the latter is arranged in the head antenna unit 24.

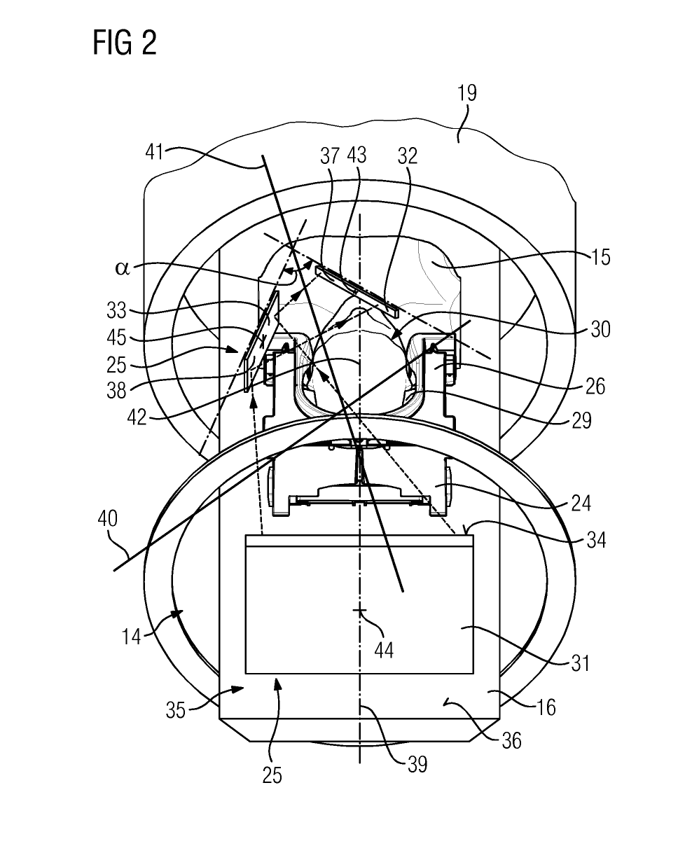

The projection unit 25 has a presentation surface unit 31, a first projection surface unit 32 and a second projection surface unit 33. A basic arrangement of a first exemplary embodiment of the projection unit 25 is represented schematically in FIG. 2, the magnet unit 11 being depicted in a cutaway view to provide a better illustration of the projection unit 25.

The presentation surface unit 31 includes a presentation surface 34 on which, in particular, picture elements are presented for stimulating the patient 15 for the purpose of functional magnetic resonance imaging. In the present exemplary embodiment, the presentation surface 34 is formed by a monitor for that purpose. Furthermore, the presentation surface unit 31 is arranged at a head-end region 35 of the patient receiving zone 14 on a housing panel of the magnetic resonance device 10 enclosing the patient receiving zone 14. Alternatively, the presentation surface 34 may also have an embodiment other than a monitor. Furthermore, the presentation surface unit 31 may be arranged at a head-end region of the patient positioning device 16. In the present exemplary embodiment, the presentation surface 34 of the presentation surface unit 31 is aligned perpendicularly to a reclining surface 36 of the patient positioning device 16.

The first projection surface unit 32 has a first projection surface 37 that is formed by a reflecting surface. The second projection surface unit 33 has a second projection surface 38 that is likewise formed by a reflecting surface. The picture elements presented on the presentation surface 34 are projected by the second projection surface unit 33 on the second projection surface 38 and presented from the latter onto the first projection surface 37.

For this purpose, the projection unit 25 has a first mirror plane 40 for projecting a picture element from the presentation surface unit 31 onto the second projection surface unit 33. The projection unit 25 additionally has a second mirror plane 41 for projecting a picture element from the second projection surface unit 33 onto the first projection surface unit 32.

The two mirror planes 40, 41 are in this case arranged relative to the presentation surface 34, the first projection surface 37 and the second projection surface 38 in such a way that an axis 39 that is defined by a center of the presentation surface 34 and a center of the first projection surface 37 intersects a normal vector of the first mirror plane 40 and a normal vector of the second mirror plane 41.

The head antenna unit 24 has a median plane 42 that divides the head antenna unit 24 into precisely a right half and a left half, the median plane 42 in this case being aligned perpendicularly to the facial opening 30 of the head antenna unit 24. The median plane 42 of the head antenna unit 24 is formed in particular by a mirror plane of symmetry of the head antenna unit 24 that divides the head antenna unit 24 into two mutually symmetrical halves or subsections. Furthermore, the median plane 42 of the head antenna unit 24 is substantially identical to a sagittal plane of the patient 15 positioned inside the head antenna unit 24. In the present exemplary embodiment, a center 43 of the first projection surface unit 32 is arranged spaced at a distance apart from the facial opening 30 of the head antenna unit 24 in the median plane 42.

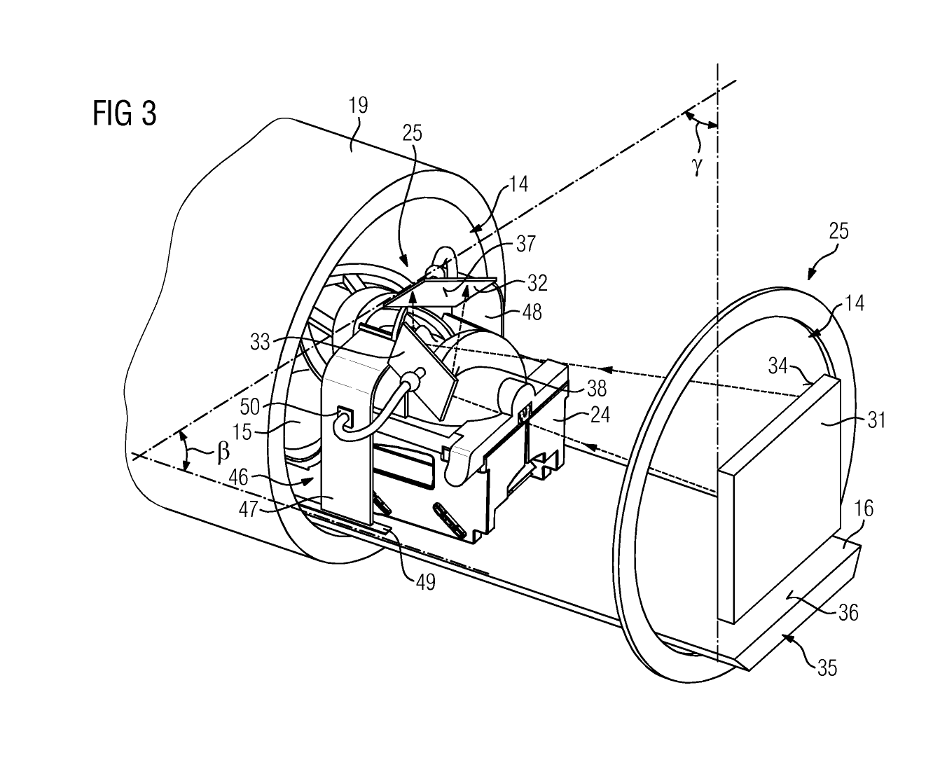

The first projection surface 37 of the first projection surface unit 32 has an angle of inclination β of 45° with respect to the reclining surface of the head antenna unit 24 or, as the case may be, to the reclining surface 36 of the patient positioning device 16 (FIG. 3). Owing to the substantially perpendicular alignment of the presentation surface 34 of the presentation surface unit 31 with respect to the reclining surface 36 of the patient positioning device 16, the first projection surface 37 of the first projection surface unit 32 also has an angle of inclination γ of 45° with respect to the presentation surface 34 (FIG. 3). This enables an upright image to be projected on the first projection surface 37, where an easily viewable image may be generated for the patient 15 positioned inside the head antenna unit 24. The first projection surface 37 has the angle of inclination γ of 45° with respect to the presentation surface 34 projected onto the median plane.

The first projection surface 37 of the first projection surface unit 32 and the second projection surface 38 of the second projection surface unit 33 are embodied separately from each other. In this case, the first projection surface 37 includes an angle of inclination of 67.5° with the second projection surface 38. An alignment of the second projection surface 38 at an angle of inclination α of 67.5° to the first projection surface 37 enables a realistic, non-inverted image and/or a true, non-inverted image of the picture elements presented on the presentation surface 34 on the first projection surface 37. However, in certain embodiments, an exact setting of the angle of inclination α of precisely 67.5° between the first projection surface 37 and the second projection surface 38 is necessary for the true, non-inverted presentation of picture elements.

The axis 39 spans a plane together with a directional vector that is aligned perpendicularly to the reclining surface of the head antenna unit 34 and/or perpendicularly to the reclining surface 36 of the patient positioning device 16, where the second projection surface unit 33 is arranged to the side of the plane. In this case, the second projection surface unit 33 is arranged completely to the side of the plane. In the present exemplary embodiment, the plane is identical to the median plane 42 of the head antenna unit 24. In the present exemplary embodiment, the second projection surface unit 33 is arranged to the left of the plane and to the left of the first projection surface unit 32 in a view from above onto the projection unit 25 and the patient positioning device 16. In an alternative embodiment, the second projection surface unit 33 may in principle also be arranged to the right of the plane or, as the case may be, to the right of the first projection surface unit 32. It is furthermore also conceivable for the plane to be oriented differently from the median plane 42 of the head antenna unit 24.

The second projection surface 38 likewise has a center 45, the center 45 of the second projection surface 38, the center 43 of the first projection surface 37 and the center 44 of the presentation surface 34 forming vertices of a triangle. An angle that is spanned from a side of the triangle that is arranged between the center 42 of the first projection surface 37 and the center 44 of the second projection surface 38 and a side of the triangle that is arranged between the center 44 of the second projection surface 38 and the center 43 of the presentation surface 34 is formed by an obtuse angle.

For arrangement of the first projection surface unit 32 and the second projection surface unit 33, the projection unit 25 has a retaining device 46. In FIG. 3, the retaining device 46 is arranged on the patient positioning device 16. The retaining device 46 includes two retaining arms 47, 48, one retaining arm 47, 48 being provided in each case for positioning one of the projection surface units 32, 33. The first retaining arm 47 is arranged at a first edge region of the patient positioning device 16. The second retaining arm 48 is arranged at a second edge region of the patient positioning device 16, the second edge region being arranged at a side of the patient positioning device 16 opposite to a side having the first edge region such that the head antenna unit 34 is arranged between the two edge regions. The first edge region, the head antenna unit 24 arranged on the patient positioning device 16 and the second edge region are arranged in succession along a width or transverse extension of the patient positioning device 16.

For an exact positioning of the first projection surface 37 and the second projection surface 38 with respect to a position and/or a viewing direction of a patient 15 arranged inside the head antenna unit 24, the two retaining arms 47, 48 of the retaining device 46 are slidably arranged on the patient positioning device 16. Toward that end, the patient positioning device 16 and also the two retaining arms 47, 48 of the retaining device 46 include a sliding-contact bearing unit 49 having a plurality of sliding-contact bearing elements that enable the retaining arms 47, 48 to be positioned at different positions along a longitudinal extension of the patient positioning device 16.

The retaining device 46 includes adjusting elements 50 for setting a position of the first projection surface unit 37 and for setting a position of the second projection surface unit 38 with respect to the reclining surface 36 or, as the case may be, with respect to the patient 15. The adjusting elements 49 may include, for example, an articulated-joint unit, in particular a ball-and-socket joint unit, and/or a flexible stand. An adjustment and/or fixing may be effected by way of a clamping action of the adjusting element 49 such that a simple and quick adjustment and/or fixing may be realized. Furthermore, other adjusting elements appearing beneficial to the person skilled in the art are conceivable in a further embodiment.

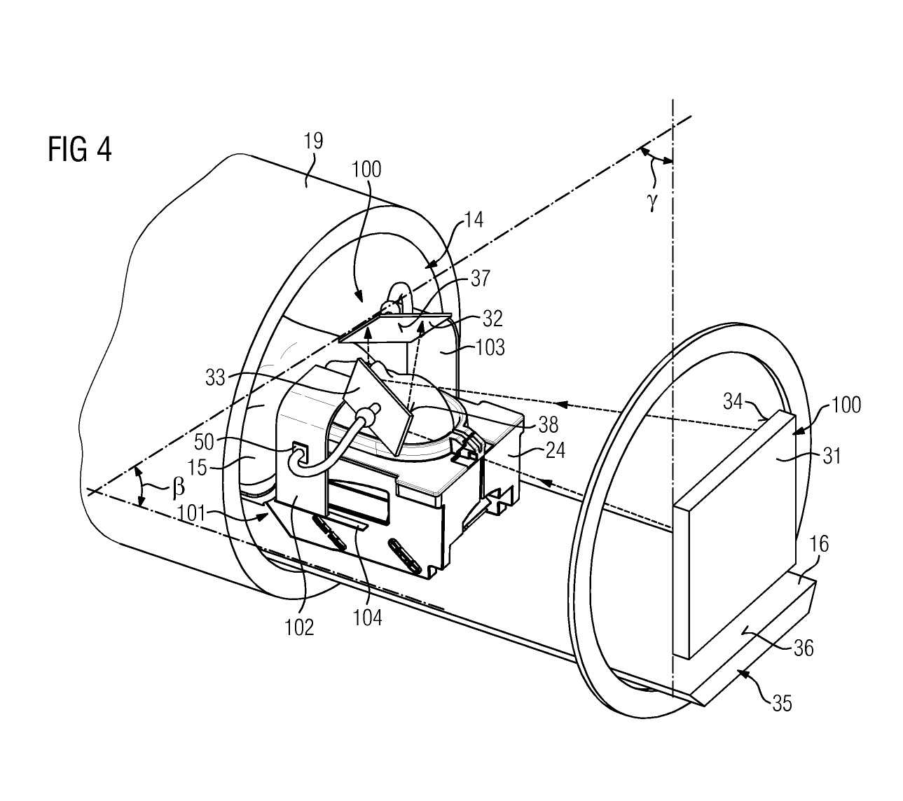

FIG. 4 depicts an alternative exemplary embodiment of the projection unit 100. Components, features and functions remaining substantially the same are labeled with the same reference numerals. The following description restricts itself essentially to the differences from the exemplary embodiment in FIGS. 2 and 3, with reference being made to the description of the exemplary embodiment in FIGS. 2 and 3 in respect of components, features and functions that remain the same.

The projection unit 100 in FIG. 4 has a first projection surface unit 32, a second projection surface unit 33 and a presentation surface unit 31. An embodiment and/or arrangement of the first projection surface unit 32, the second projection surface unit 33 and the presentation surface unit 31 corresponds to the statements made with reference to FIGS. 2 and 3.

The projection unit 100 additionally has a retaining device 101 that in the present exemplary embodiment is arranged on the head antenna unit 24. The retaining device 101 includes two retaining arms 102, 103, one retaining arm 102, 103 being provided in each case for positioning one of the projection surface units 32, 33. The first retaining arm 102 is arranged at a first edge region of the head antenna unit 24. The second retaining arm 103 is arranged at a second edge region of the head antenna unit 24. The second edge region is arranged at a side of the head antenna unit 24 opposite to a side having the first edge region along a transverse extension of the head antenna unit 24.

For an exact positioning of the first projection surface 37 and the second projection surface 38 with respect to a position and/or a viewing direction of a patient 15 arranged inside the head antenna unit 24, the retaining device 101, (e.g., the two retaining arms 102, 103), is slidably arranged on the head antenna unit 24. Toward that end, the head antenna unit 24 and also the retaining device 101, (e.g., the two retaining arms 102, 103), include a sliding-contact bearing unit 104 having sliding-contact bearing elements.

A further embodiment of the retaining device 101 corresponds to the statements made with reference to FIG. 3, in particular, with regard to an embodiment of adjusting elements 49 for setting a position of the first projection surface unit 32 and/or for setting a position of the second projection surface unit 33.

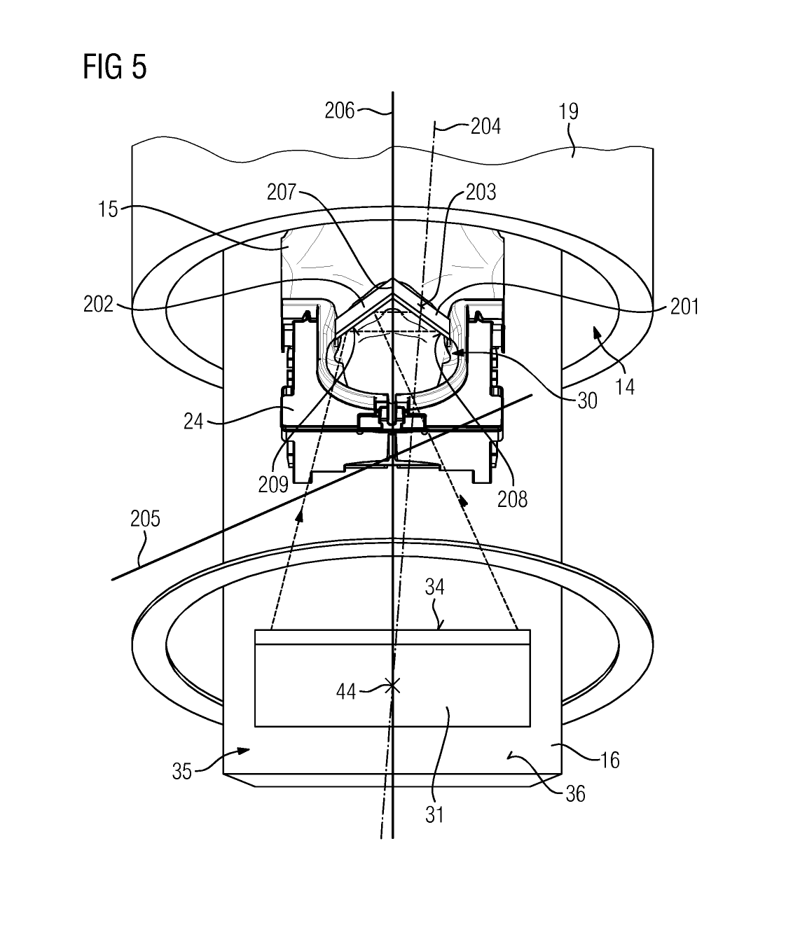

Alternative exemplary embodiments of the projection unit are depicted in FIGS. 5 to 7. Components, features, and functions remaining substantially the same are labeled with the same reference numerals. The following description restricts itself essentially to the differences from the exemplary embodiment in FIGS. 2 to 4, with reference being made to the description of the exemplary embodiment in FIGS. 2 to 4 in respect of components, features and functions that remain the same.

The projection unit 200 in FIG. 5 depicts a basic arrangement of a presentation surface unit 31, a first projection surface unit 201, and a second projection surface unit 202. The embodiment and arrangement of the presentation surface unit 31 correspond to the statements made with reference to FIGS. 2 to 4.

The first projection surface unit 201 has a center 203 and the presentation surface unit 31 also has a center 44 that together define an axis 204. The projection unit 200 has a first mirror plane 205 for projecting a picture element from the presentation surface unit 31 onto the second projection surface unit 202. The projection unit 200 additionally has a second minor plane 206 for projecting a picture element from the second projection surface unit 202 onto the first projection surface unit 201. The two minor planes 205, 206 are, in this case, arranged with respect to the presentation surface 34, a first projection surface 208 of the first projection surface unit 201, and a second projection surface 209 of the second projection surface unit 202 in such a way that the axis 204 intersects a normal vector of the first minor plane 205 and a normal vector of the second mirror plane 206.

In the present exemplary embodiment, the first projection surface unit 201 and the second projection surface unit 202 are arranged directly adjacent to each other. The head antenna unit 24 has a median plane 42, a boundary line 207 between the first projection surface unit 201, and the second projection surface unit 202 being arranged spaced at a distance apart from the facial opening 30 of the head antenna unit 24 in the median plane 42. In the present exemplary embodiment the median plane 42 of the head antenna unit is identical to the second minor plane 206.

In the present exemplary embodiment, an angle of inclination of the first projection surface 208 of the first projection surface unit 201 with respect to the presentation surface 34 and the reclining surface 36 of the patient positioning device 16 corresponds to the statements made with reference to FIGS. 2 to 4. Owing to the directly adjacent arrangement of the first projection surface unit 201 with the second projection surface unit 202, the second projection surface 209 of the second projection surface unit 20 also has an angle of inclination β, γ of 45° to the presentation surface 34 of the presentation surface unit 31 and to the reclining surface 36 of the patient positioning device 16 or, as the case may be, to the reclining surface of the head antenna unit 24.

The first projection surface 208 includes an angle of inclination α of 90° with the second projection surface 209. The arrangement of the two projection surfaces 208, 209 relative to each other enables an upright and realistic, non-inverted and/or true, non-inverted image projection on the first projection surface unit 201. However, in certain embodiments, an exact setting of the angle of inclination α of precisely 90° between the first projection surface 208 and the second projection surface 208 is necessary for the true, non-inverted imaging of picture elements in the case of adjacent projection surfaces 208, 209. A further embodiment and arrangement of the first projection surface unit 201 and the second projection surface unit 202 corresponds to the statements made with reference to FIGS. 2 to 4.

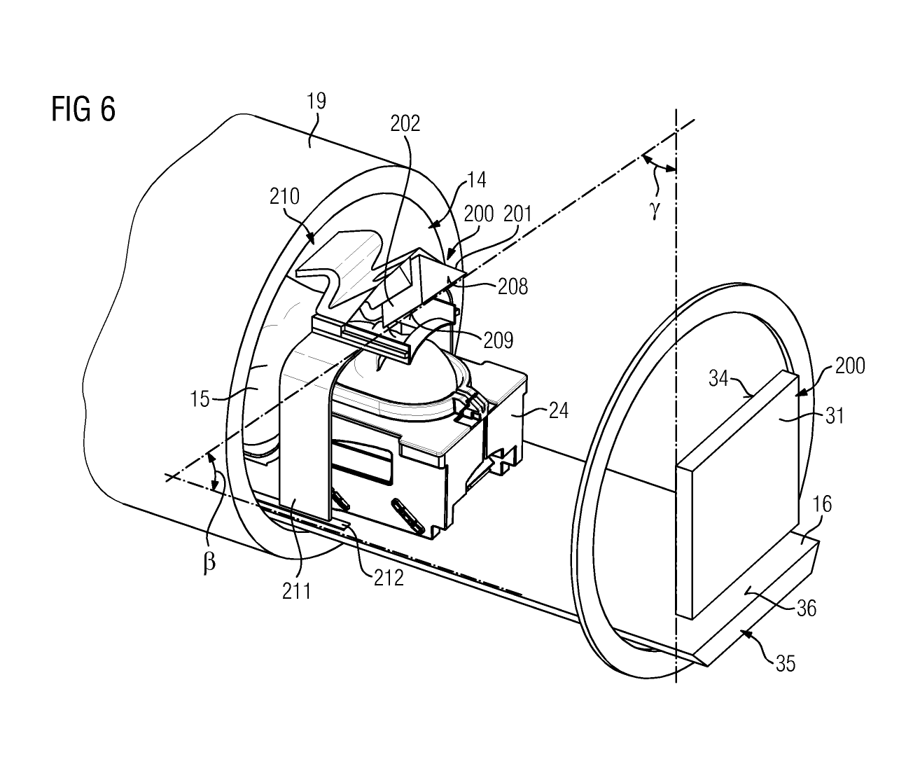

For arrangement of the first projection surface unit 208 and the second projection surface unit 209, the projection unit 25 has a retaining device 210. In FIG. 6, the retaining device 210 is arranged on the patient positioning device 16, while a depiction of the mirror planes 205, 206 is dispensed with in FIG. 5 for clarity of illustration reasons. For an exact positioning of the first projection surface 208 and the second projection surface 209 with respect to a position and/or a viewing direction of a patient 15 arranged inside the head antenna unit 24, the retaining device 210 is slidably arranged on the patient positioning device 16. Toward that end, the patient positioning device 16 and also the retaining device 210 include a sliding-contact bearing unit 212 having a plurality of sliding-contact bearing elements. Owing to the adjacent arrangement of the first projection surface unit 201 and the second projection surface unit 202, the retaining device 210 has a single retaining arm 211 for positioning the first projection surface unit 201 and the second projection surface unit 202, which retaining arm 211 is arranged at an edge region of the patient positioning device 16. A further embodiment of the retaining device 210, in particular with regard to a slidability of the retaining arm 211 and an embodiment of adjusting elements of the retaining device 210, corresponds to the statements made with reference to FIGS. 2 to 3.

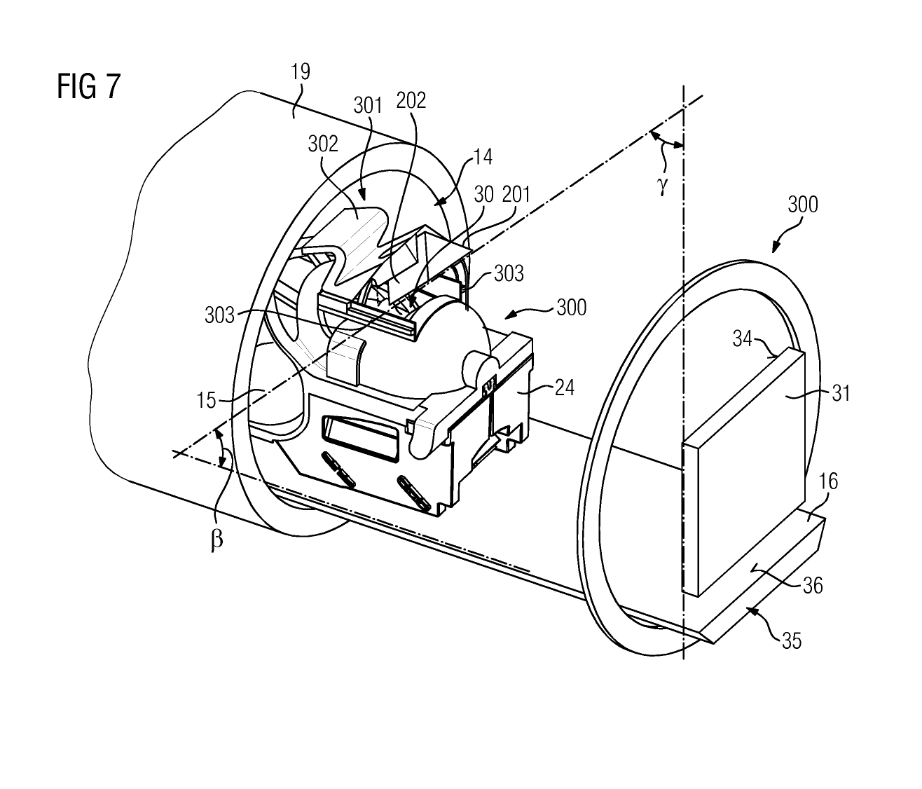

An alternative exemplary embodiment of the projection unit 300 is depicted in FIG. 7. Components, features, and functions remaining substantially the same are labeled with the same reference numerals. The following description restricts itself essentially to the differences from the exemplary embodiment in FIGS. 5 to 6, with reference being made to the description of the exemplary embodiment in FIGS. 5 to 6 in respect of components, features, and functions that remain the same.

The projection unit 300 in FIG. 7 has a first projection surface unit 201, a second projection surface unit 202, and a presentation surface unit 31. An embodiment and/or arrangement of the first projection surface unit 201, the second projection surface unit 202 and the presentation surface unit 31 corresponds to the statements made with reference to FIGS. 5 and 6.

The projection unit 300 additionally has a retaining device 301 that in the present exemplary embodiment is arranged on the head antenna unit 24. The retaining device 301 includes a single retaining arm 302, the retaining arm 302 being provided for positioning the two projection surface units 201, 202. The retaining arm 302 is movably, in particular slidably, mounted on the head antenna unit 24 by way of a sliding-contact bearing unit 303 that is arranged at opposite edge regions of the head antenna unit 24, the two edge regions at least partially bordering a facial region 30 of the head antenna unit 24 on opposite sides. An exact positioning of a first projection surface 208 of the first projection surface unit 201 and of a second projection surface 209 of the second projection surface unit 202 with respect to a position and/or a viewing direction of a patient 15 arranged inside the head antenna unit 24 is possible by the sliding-contact bearing unit 303. For this purpose, the sliding-contact bearing unit 303 includes sliding-contact bearing elements on the head antenna unit 24 and on the retaining arm 302.

A further embodiment of the retaining device 301 corresponds to the statements made with reference to FIG. 6, in particular, with regard to an embodiment by adjusting elements for setting a position of the first projection surface unit 201 and/or for setting a position of the second projection surface unit 202.

Although the invention has been illustrated and described in greater detail on the basis of the exemplary embodiments, it is not limited by the disclosed examples and other variations may be derived therefrom by the person skilled in the art without leaving the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A magnetic resonance device, the device comprising:
a head antenna unit configured for acquiring magnetic resonance signals during a magnetic resonance measurement; and
a projection unit comprising:
a presentation surface unit for presenting picture elements;
a first projection surface unit for presenting a projection of the picture elements presented on the presentation surface unit, wherein the first projection surface unit has a center and the presentation surface unit has a center, the center of the first projection surface unit defining an axis together with the center of the presentation surface unit;
a second projection surface unit;
a first mirror plane for projecting a picture element from the presentation surface unit onto the second projection surface unit; and
a second mirror plane for projecting a picture element from the second projection surface unit onto the first projection surface unit, wherein the axis intersects a normal vector of the first mirror plane and/or a normal vector of the second mirror plane.

2. The magnetic resonance device as claimed in claim 1, wherein the first projection surface unit comprises a first projection surface, the first projection surface having an angle of inclination of 45° with respect to (1) a reclining surface of the head antenna unit, (2) a reclining surface of a patient positioning device, or (3) the reclining surface of the head antenna unit and the reclining surface of a patient positioning device.

3. The magnetic resonance device as claimed in claim 1, wherein the first projection surface unit comprises a first projection surface and the presentation surface unit comprises a presentation surface, wherein the first projection surface has an angle of inclination of 45° with respect to the presentation surface.

4. The magnetic resonance device as claimed in claim 3, further comprising a patient positioning device comprising a reclining surface,
wherein the axis spans a plane with a directional vector that is aligned perpendicularly to a reclining surface of the head antenna unit or perpendicularly to the reclining surface of the patient positioning device, and
wherein the second projection surface unit is arranged to the side of the plane.

5. The magnetic resonance device as claimed in claim 1, further comprising a patient positioning device comprising a reclining surface,
wherein the axis spans a plane with a directional vector that is aligned perpendicularly to a reclining surface of the head antenna unit or perpendicularly to the reclining surface of the patient positioning device, and
wherein the second projection surface unit is arranged to the side of the plane.

6. The magnetic resonance device as claimed in claim 1, wherein the first projection surface unit is separate from the second projection surface unit.

7. The magnetic resonance device as claimed in claim 1, wherein the first projection surface unit comprises a first projection surface and the second projection surface unit comprises a second projection surface, wherein the first projection surface has an angle of inclination of 67.5° with respect to the second projection surface.

8. The magnetic resonance device as claimed in claim 1, wherein the head antenna unit comprises a facial opening and a median plane, wherein the center of the first projection surface unit is arranged spaced at a distance apart from the facial opening of the head antenna unit in the median plane.

9. The magnetic resonance device as claimed in claim 1, wherein the head antenna unit comprises a facial opening and a median plane, and
wherein the first projection surface unit and the second projection surface unit are arranged directly adjacent to each other, wherein a boundary line between the first projection surface unit and the second projection surface unit is arranged spaced at a distance apart from the facial opening of the head antenna unit in the median plane.

10. The magnetic resonance device as claimed in claim 9, wherein the first projection surface unit comprises a first projection surface and the second projection surface unit comprises a second projection surface, wherein the first projection surface includes an angle of inclination of 90° with respect to the second projection surface.

11. The magnetic resonance device as claimed in claim 1, further comprising a patient positioning device and a patient receiving zone,
wherein the presentation surface unit is arranged at an end region of the patient positioning device, an end region of the patient receiving zone, or the end region of the patient positioning device and the end region of the patient receiving zone.

12. The magnetic resonance device as claimed in claim 1, wherein the projection unit comprises a retaining device for positioning the first projection surface unit, the second projection surface unit, or the first projection surface unit and the second projection surface unit,
wherein the retaining device is arranged on the head antenna unit.

13. The magnetic resonance device as claimed in claim 12, wherein the retaining device comprises at least one adjusting element for setting a position of the first projection surface unit, the second projection surface unit, or the first projection surface unit and the second projection surface unit.

14. The magnetic resonance device as claimed in claim 12, wherein the retaining device is slidably arranged on the head antenna unit.

15. The magnetic resonance device as claimed in claim 14, wherein the retaining device comprises at least one adjusting element for setting a position of the first projection surface unit, the second projection surface unit, or the first projection surface unit and the second projection surface unit.

16. The magnetic resonance device as claimed in claim 1, further comprising a patient positioning device,
wherein the projection unit has a retaining device for positioning the first projection surface unit, the second projection surface unit, or the first projection surface unit and the second projection surface unit, and
wherein the retaining device is arranged on the patient positioning device.

17. The magnetic resonance device as claimed in claim 16, wherein the retaining device comprises at least one adjusting element for setting a position of the first projection surface unit, the second projection surface unit, or the first projection surface unit and the second projection surface unit.

18. The magnetic resonance device as claimed in claim 16, wherein the retaining device is slidably arranged on the patient positioning device.

19. The magnetic resonance device as claimed in claim 18, wherein the retaining device comprises at least one adjusting element for setting a position of the first projection surface unit, the second projection surface unit, or the first projection surface unit and the second projection surface unit.

20. The magnetic resonance device as claimed in claim 16, wherein the patient positioning device comprises a reclining surface,
wherein the axis spans a plane with a directional vector that is aligned perpendicularly to a reclining surface of the head antenna unit or perpendicularly to the reclining surface of the patient positioning device, and
wherein the second projection surface unit is arranged to the side of the plane.
